(19) United States
(12) Patent Application Publication
(10) Pub. No.: US 2025/0032789 A1
(43) Pub. Date: Jan. 30, 2025

(54) ELECTRODE CONFIGURATION AND METHOD FOR AURICULAR VAGAL NERVE STIMULATION

(71) Applicant: COGNIGUARD MEDICAL HOLDINGS LIMITED, Nicosia (CY)

(72) Inventors: Tamir Ben David, Tel Aviv (IL); Adam Broncel, Lodz (PL); Raanan Gefen, Re'ut (IL)

(73) Assignee: COGNIGUARD MEDICAL HOLDINGS LIMITED, Nicosia (CY)

(21) Appl. No.: 18/716,940

(22) PCT Filed: Dec. 20, 2022
(86) PCT No.: PCT/IL2022/051354

§ 371 Date: Jun. 6, 2024

Related U.S. Application Data
(60) Provisional application No. 63/291,469, filed on Dec. 20, 2021.

(51) Int. Cl. A61N 1/36; A61N 1/04

(52) U.S. Cl. CPC A61N 1/36025; A61N 1/0456; A61N 1/0472; A61N 1/36036

(57) ABSTRACT

Disclosed herein is a device for auricular vagal nerve stimulation in a. subject, said device having an electrode configuration including at least one electrode contact operative to be adhered to a first portion of inner skin surface of an ear Concha of the subject, and at least one return electrode with an opposite polarity operative to be adhered to a second portion of the inner skin surface.

### FIELD

The invention relates to the field of vagal nerve stimulation.

### BACKGROUND

Vagal nerve stimulation is a known solution to activate cerebral and visceral structures in individuals who suffer from various neurologic and somatic disorders. Several solutions already exist, for example, invasive implantable devices, and non-invasive transdermal stimulation with several configurations like electrical stimulation of the cervical vagus, using an electrode placed at the neck over the cervical vagus, etc. Alternatively, a noninvasive configurations to facilitate activation of the auricular branch of the vagus nerve (“ABVN”) using in-ear electrodes. Stimulation of ABVN typically activates myelinated neuron fibers only from A-group sensory axons.

Stimulation of the vagus nerve may affect many physiological mechanisms. Due to the importance of the vagus nerve as the main nerve that activates the parasympathetic nervous system in the system's descending paths to various internal organs including the heart, the intestines, the bladder, and the endocrine glands. Furthermore, the vagus nerve, through ascending neurons, effects the brain steam nuclei's and induces a response from higher cerebral structures.

Invasive vagal nerve stimulation (“VNS’) is used as a standard of care for drug refractory epilepsy patients. In such invasive treatment, the left cervical vagus nerve is exposed, followed by placing an electrode around the nerve. This electrode is connected to an active device, typically implanted in the left upper chest area. The device is typically programmed to deliver alternating stimulation, typically 30 seconds of active stimulation followed by 5 minutes of no stimulation. Such use of invasive treatment involves significant risks posed by the surgical intervention and mechanical irritation of nerve fiber, with particular risks related to nerve damage and risk of infection at the locations of the electrode, lead and device.

Electrical activation of the vagus nerve is known to activate the brain in various areas, including the Hippocampus as observed by Broncel (Broncel A, Bocian R, Klos-Wojtczak P, Konopacki J 2017). Vagus nerve stimulation produces a hippocampal formation theta rhythm in anesthetized rats, 2017 Aug. 30) and may provide a wide-range of therapeutic applications, this may be particularly beneficial when providing noninvasive and safe VNS treatment.

FIG. 1A is a front view schematic illustration of an ear 5 including an outer portion of the ear 8, ear canal 10. Cavum Conchae 11 and Cymba Conchae 12. Ear 5 includes a schematic representation of auricular branches 13 of the Vagus nerve.

FIG. 1B is a rear view schematic illustration of ear 5 showing a rear area 6 of ear 5, a rear head portion 30 of a subject and an interface portion 35, which is where ear 5 connects to a head of the user.

### SUMMARY

The following embodiments and aspects thereof are described and illustrated in conjunction with systems, tools and methods which are meant to be exemplary and illustrative, not limiting in scope.

There is provided, in accordance with an embodiment, a device for auricular vagal nerve stimulation in a subject, the device having an electrode configuration including at least one electrode contact operative to be adhered to a first portion of inner skin surface of an ear Cavum Concha of the subject, and at least one return electrode with an opposite polarity operative to be adhered to a second portion of a skin surface of the subject.

In some embodiments, the at least one return electrode comprises a bow shape to facilitate positioning the at least second electrode within the ear canal.

In some embodiments, the at least one return electrode comprises a circular shape to facilitate positioning the at least second electrode within the ear canal.

In some embodiments, the at least one electrode comprises a surface area within a range of 0.5-2 square centimeters.

In some embodiments, the at least one electrode comprises a surface area within a range of 0.1-5 square centimeters.

In some embodiments, the at least one electrode comprises a surface area within a range of 0.2-1 square centimeters.

In some embodiments, the at least one electrode is customizable to fit ear Cavum Concha area.

In some embodiments, the at least first electrode comprises a plurality of peripheral indentations.

In some embodiments, the at least first electrode and the at least second electrode are flexible.

In some embodiments, the at least first electrode is a Transcutaneous electrical nerve stimulation (TENS) electrode.

In some embodiments, the at least second electrode is a Transcutaneous electrical nerve stimulation (TENS) electrode.

In some embodiments, the at least first electrode is operative to provide stimulation while a subject is asleep.

In some embodiments, the second electrode is positioned on the skin surface of rear region of the ear.

In some embodiments, the second electrode is positioned on the skin surface of rear region of a head of the subject.

In some embodiments, the second electrode is positioned on the skin surface at an interface between the ear and a head of the subject.

In some embodiments, the first electrode comprises a surface area greater than 50% of the skin surface of the Cavum Concha.

There is further provided, in accordance with an embodiment, a device for auricular vagal nerve stimulation in a subject, the device having an electrode configuration including at least one electrode contact operative to be adhered to a first portion of inner skin surface of an car of the subject, and at least one return electrode with an opposite polarity operative to be adhered on a skin area of a rear region of the ear.

In some embodiments, the at least one electrode comprises a surface area within a range of 0.1-5 square centimeters.

In some embodiments, the at least one electrode is customizable to fit regions of ear.

In some embodiments, the at least first electrode comprises a plurality of peripheral indentations.

In some embodiments, the at least first electrode and the at least second electrode are flexible.

In some embodiments, the at least first electrode is a Transcutaneous electrical nerve stimulation (TENS) electrode.

There is further provided, in accordance with an embodiment, a device for auricular vagal nerve stimulation in a subject, the device having an electrode configuration including at least one electrode contact operative to be adhered to a first portion of inner skin surface of an ear of the subject, and at least one return electrode with an opposite polarity operative to be adhered to an interface area between the ear and a head of the subject.

In some embodiments, the at least first electrode comprises a surface area within a range of 0.1-5 square centimeters.

In some embodiments, the at least first electrode is customizable to fit regions of ear.

In some embodiments, the at least first electrode comprises a plurality of peripheral indentations.

In some embodiments, the at least first electrode and the at least second electrode are flexible.

In some embodiments, the at least first electrode is a Transcutaneous electrical nerve stimulation (TENS) electrode.

In some embodiments, the at least second electrode is a Transcutaneous electrical nerve stimulation (TENS) electrode.

There is further provided, in accordance with an embodiment, a method of positioning an electrode on a skin surface area of an ear, the method including cleaning a skin surface area, positioning the electrode on the skin surface area, measuring an impedance to determine whether the electrode is properly positioned, connecting the electrode to a stimulator, and delivering an electrical stimulation to the ear.

In some embodiments, the electrical stimulation is administered on when the impedance is less than a predetermined value.

In some embodiments, the method further includes further cleaning the skin surface area and/or repositioning the electrode when the impedance is greater than a predetermined value.

In some embodiments, the skin surface area is a Cavuum Conchae of the ear.

In some embodiments, the method further includes measuring the surface area of the ear, and selecting an electrode size and shape so as to fit the electrode size to the ear.

In some embodiments, the method further includes manufacturing the electrode at the measured electrode size.

In addition to the exemplary aspects and embodiments described above, further aspects and embodiments will become apparent by reference to the figures and by study of the following detailed description.

### BRIEF DESCRIPTION OF THE FIGURES

Some non-limiting exemplary embodiments or features are illustrated in referenced figures. Dimensions of components and features shown in the figures are generally chosen for convenience and clarity of presentation and are not necessarily shown to scale. The figures are listed below.

FIGS. 1A-1B are schematic illustrations of an ear anatomy, according to certain exemplary embodiments;

FIG. 2 is a schematic illustration of a bipolar configuration of electrodes for the ear of FIG. 1, according to certain exemplary embodiments;

FIGS. 3A-3C are schematic illustrations of a bipolar configuration of a first electrode positioned within a Conchae of the ear of FIG. 1 and a second electrode positioned within an ear canal of the ear, according to certain exemplary embodiments:

FIG. 4 is a schematic illustration of a bipolar configuration figuration of a first electrode positioned within the Cavum Conchae of the ear of FIG. 1 and a second electrode positioned within a Cymba Conchae of the ear, according to certain exemplary embodiments;

FIGS. 5A-5B are schematic illustrations of a bipolar configuration figuration of a first electrode positioned within the Cavum Conchae of the ear of FIG. 1 and a second electrode positioned along an outer portion of the ear, according to certain exemplary embodiments;

FIGS. 6A-6B are schematic illustrations of a bipolar configuration figuration of a first electrode positioned within the Cavum Conchae of the ear of FIG. 1 and a second electrode positioned on an outer head portion of a subject adjacent to the ear, according to certain exemplary embodiments;

FIGS. 7A-7B are schematic illustrations of a bipolar configuration figuration of a first electrode positioned within the Cavum Conchae of the ear of FIG. 1 and a second electrode positioned on an interface between ear and the outer head portion of the subject adjacent, according to certain exemplary embodiments;

FIGS. 8A-8D schematically illustrate a plurality of shapes of the in ear electrodes, according to certain exemplary embodiments:

FIG. 9 is a schematic illustration of a cross section side view of an electrode within a Cavum Conchae of the ear of FIG. 1, according to certain exemplary embodiments:

FIG. 10 is a schematic illustration of an in-ear electrode including peripheral indentations to facilitate positioning of the in-ear electrode on a concave surface, according to certain exemplary embodiments;

FIGS. 11A-11D are schematic illustrations of a plurality of in-ear electrode configurations, each in-car electrode including peripheral indentations to facilitate positioning of the in ear electrode on a concave surface, according to certain exemplary embodiments;

FIG. 12 is a schematic illustration of an in-ear electrode including a uniform current density inner wire net configuration, according to certain exemplary embodiment:

FIG. 13 is a schematic illustration of the in-ear electrode of FIG. 12 further including peripheral indentations, according to certain exemplary embodiments:

FIG. 14 is a schematic illustration of an in-ear electrode including a soft wire, according to certain exemplary embodiments;

FIG. 15 is a schematic illustration of the in-ear electrode of FIG. 14 further including a finger attachment that facilitates proper positioning of the electrode in the Concha, according to certain exemplary embodiments;

FIGS. 16A-16B are schematic illustrations of an over ear device connected to an in-car electrode and an out-ear electrode, according to certain exemplary embodiments;

FIGS. 17A-17D are schematic illustrations of a behind the ear device connected to an out-car electrode, according to certain exemplary embodiments;

FIGS. 18A-18B are schematic illustrations of a behind the head device connected to an out-ear electrode, according to certain exemplary embodiments; and

FIGS. 19A-19B are schematic illustrations of a behind the head device connected to an in-ear electrode and an out-ear electrode, according to certain exemplary embodiments.

Identical, duplicate or equivalent, or similar structures, elements, or parts that appear in one or more drawings are generally labeled with the same reference numeral, optionally with an additional letter or letters to distinguish between similar entities or variants of entities, and may not be repeatedly labeled and/or described. Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

References to previously presented elements are implied without necessarily further citing the drawing or description in which they appear.

### DETAILED DESCRIPTION

Disclosed herein is an electrode configuration and method for auricular vagal nerve stimulation to facilitate the treatment of patients who suffer from neurological disorders, according to certain exemplary embodiments. The stimulation can facilitate the treatment of neurological disorders such as Alzheimer's, Parkinson's, tremor, depression, migraine, headache, peripheral pain, attention deficit disorder (“ADD”), attention deficit and hyperactivity disorder (“ADHD”), sleeping disorders, cognitive dysfunctions, sexual dysfunctions and the like. The treatment is achieved through activation of the nerve system using various techniques, such as, electrical stimulation of the Auricular Branch of the Vagus Nerve (“ABVN”), sensory stimulations, cognitive stimulations or the like. The ABVN is an afferent sensory channel to the brain that branches into the main Vagus nerve. Patients who have undergone invasive VNS have reported on sensations from the ear but only during stimulation. Transcutaneous vagal nerve stimulation (“tVNS”) can be delivered through the ABVN, because the nerve branches are close to the skin.

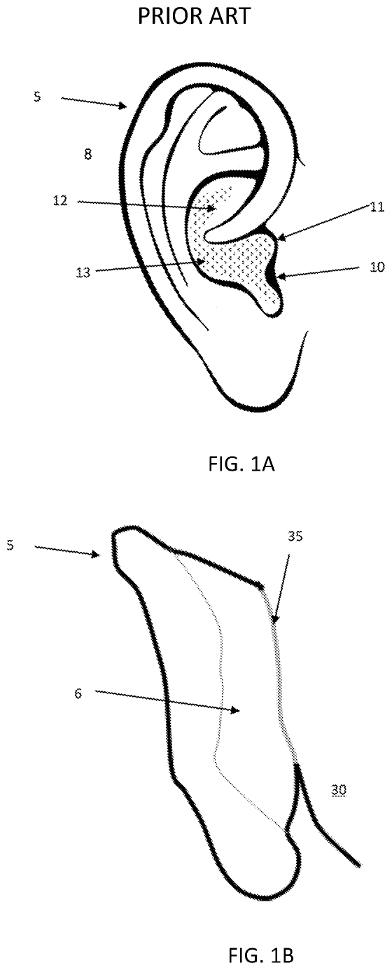

In some embodiments, the electrode having a predetermined shape can provide an effective stimulation to the ABVN adjacent 13 (FIG. 1) to the cavum Conchae 11 (FIG. 1), cymba Conchae 12 (FIG. 1). Anthelix, external auditory meatus, and the like. In some cases, the stimulation can also be performed at the eminence of the Conchae and eminence of triangular fossa of the ear 5 (FIG. 1).

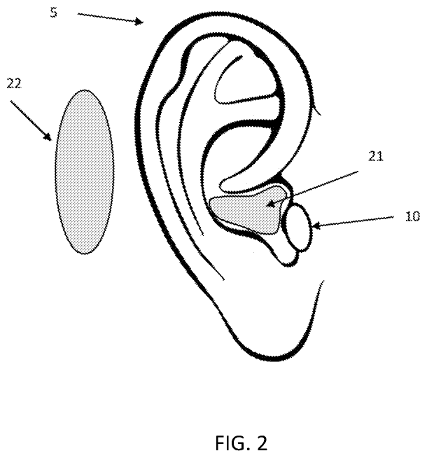

Referring to FIG. 2, schematically illustrates an electrode configuration of electrodes 21, 22 for the ear 5 (FIG. 1), according to certain exemplary embodiments. In some embodiments, the geometrical configuration of the electrodes can include a large surface electrode 21, for adhering to a skin portion in a flat area of Cavum Conchae 11 (FIG. 1). For example, the large surface area of the large surface electrode 21 can be within a range of 0.1-5 square centimeters(“cm²”). In some embodiments, the large surface area can be within a range of 0.2-1 cm² or in some embodiments the large surface area can be within a range of 0.5-2 cm². In some embodiments, the electrode configuration can include a bipolar electrode configuration having a second electrode having an opposite polarity of first electrode 21. In some embodiments, the second electrode can be positioned on a portion of skin surface area of ear 5, having a surface area within a range of 0.2-10 cm².

In some embodiments, first electrode 21 can have a predetermined size within a percentage range of a flat surface area of Cavuum Conchae 11. For example, the percentage of the predetermined size can be within a range of 10% to 90% of the surface area. In some embodiments, first electrode 21 can have a surface area greater and 20% or 50% of the Cavuum Conchae 11.

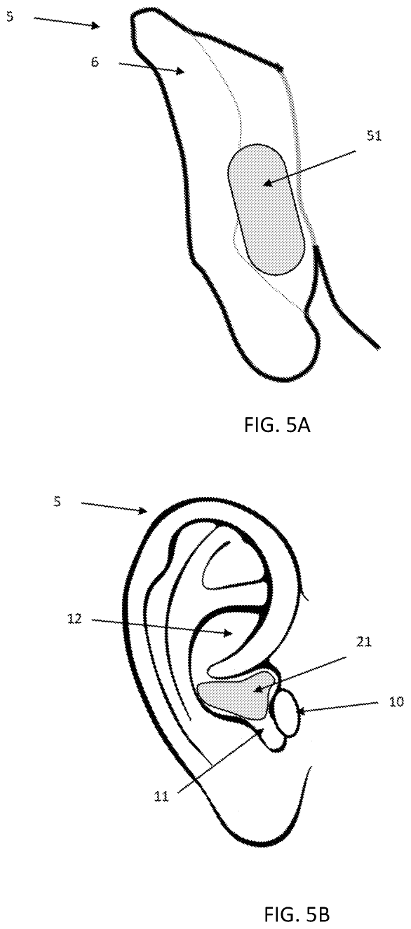

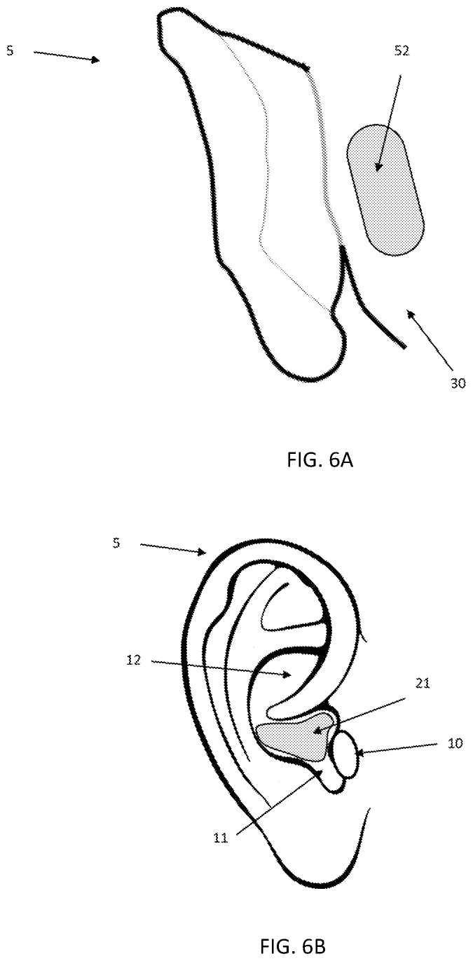

The second electrode can include an out of ear electrode 22 configured to be positioned behind the ear as described herein in conjunction with FIGS. 5A-5B, behind a head 30 (FIG. 6A) as described herein in conjunction with FIGS. 6A-6B, behind a neck of the subject, or the like. In some exemplary embodiments, electrodes 21, 22 can be Transcutaneous electrical nerve stimulation (“TENS”) electrodes. In some embodiments, electrodes 21, 22 can be flexible to facilitate positioning of electrodes 21, 22 on ear 5. In some embodiments, electrodes 21, 22 can be composed of carbon rubber, or the like.

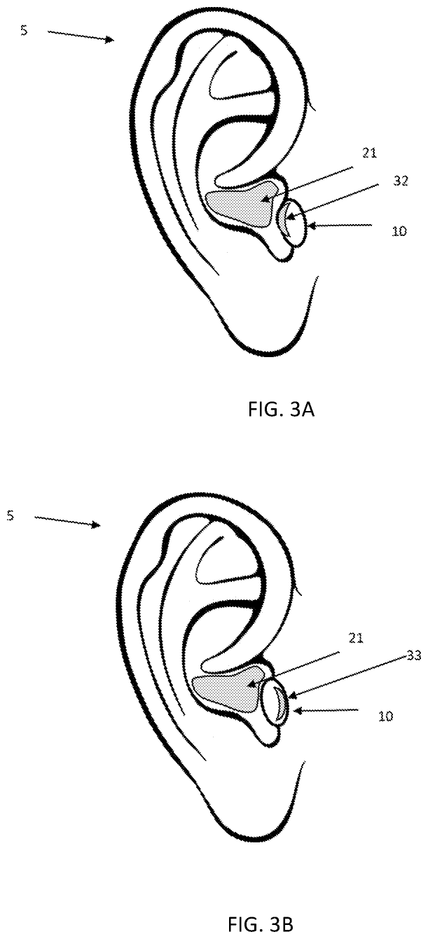

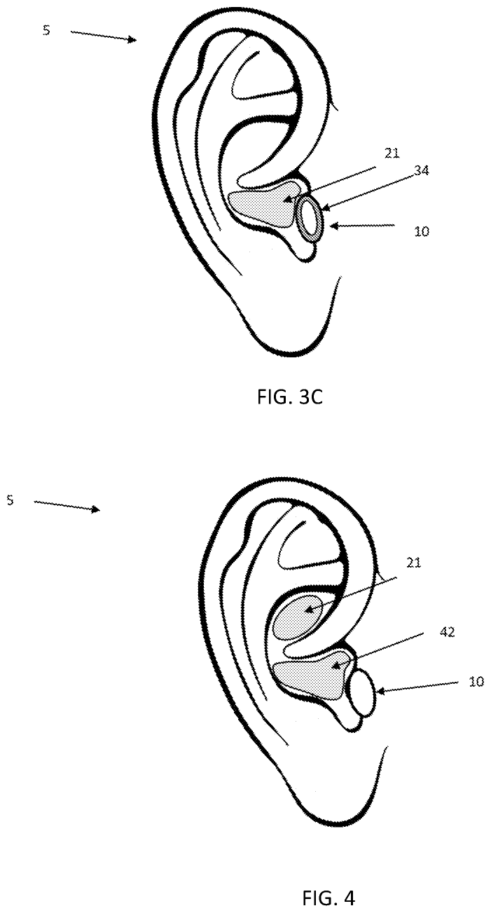

Referring to FIG. 3A, schematically illustrating a bipolar configuration of a first electrode 21 positioned within Cavum Conchae 11 (FIG. 1) and a second in-ear electrode 32 having a bow shape positioned along a first wall within ear canal 10, according to certain exemplary embodiments.

FIG. 3B schematically illustrates a bipolar configuration of first electrode 21 positioned within Cavum Conchae 11 (FIG. 1) and a second in-ear electrode 33 having a bow shape positioned along a second wall within ear canal 10, according to certain exemplary embodiments.

Referring to FIG. 3C, schematically illustrating a bipolar configuration of first electrode 21 positioned within Cavum Conchae 11 (FIG. 1) and a second in-ear electrode 34 having a circular shape positioned ear canal 10, according to certain exemplary embodiments.

FIG. 4 schematically illustrates a bipolar configuration of first electrode 21 positioned within the Cavum Conchae 11 and a second electrode 42 positioned within cymba Conchae 12 (FIG. 1), according to certain exemplary embodiments.

FIGS. 5A-5B schematically illustrate a bipolar configuration of first electrode 21 positioned within the Cavum Conchae 11 and a second electrode 51 positioned along an outer portion 6 of ear 5, according to certain exemplary embodiments.

FIGS. 6A-6B schematically illustrate a bipolar configuration of first electrode 21 positioned within the Cavum Conchae 11 and a second electrode 52 positioned along an outer head portion 30 of a subject, according to certain exemplary embodiments. Second electrode 52 is positioned along outer head portion 30 adjacent to ear 5.

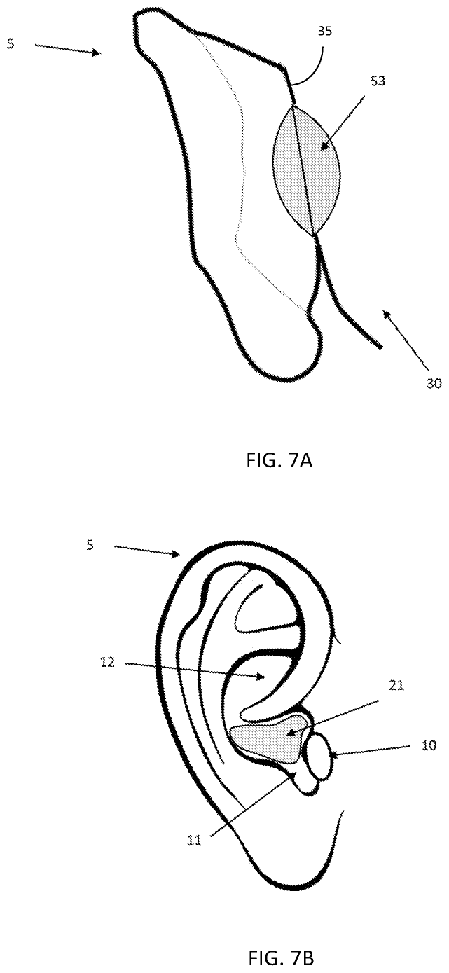

FIGS. 7A-7B schematically illustrate a bipolar configuration of first electrode 21 positioned within the Cavum Conchae 11 and a second electrode 53 positioned on an interface 35 between ear 5 and outer head portion 30, according to certain exemplary embodiments.

The shape of Cavumn Conchae 11 area can significantly vary between different people, therefore the electrode geometrical configuration can be customized to personally design to fit the regions of ear 5. Such personalization can include measuring the patient flat Cavumn Conchae 11 area, using length or area measurement means. Such means may do the measurement of the Conchae area on the patient's ear picture. Once the shape of the Cavum Conchae is detected, a special Cavum Conchae electrode is manufactured adjusting the electrode shape to the flat Conchae area measured from the patient. Such measurement can be done for both left and right ears separately. In some cases, once the shape of the Cavum Conchae is detected, a personalized electrode selection is done from a catalog of existing in ear electrodes having different sizes and shapes.

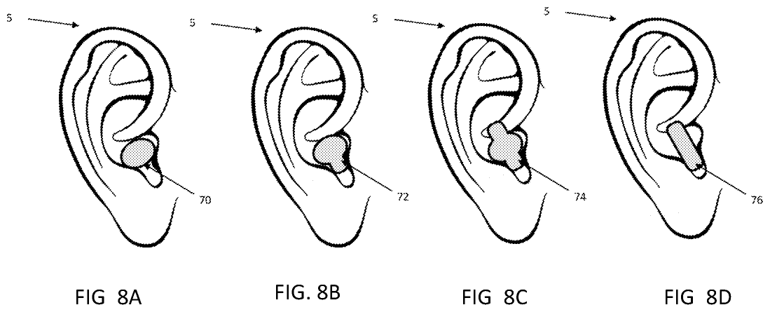

FIG. 8A shows schematically illustrate an in-ear electrode 70 having a circular shape to facilitate positioning of in-ear electrode 70 within ear canal 10 (FIG. 1), according to certain exemplary embodiments.

FIG. 8B shows an in-ear electrode 72 having a second shape to facilitate positioning of in-ear electrode 72 within ear canal 10 (FIG. 1), according to certain exemplary embodiments.

FIG. 8C shows schematically illustrate an in-ear electrode 74 having a linear shape to facilitate positioning of in-ear electrode 74 to cover ear canal 10 (FIG. 1), according to certain exemplary embodiments.

FIG. 8D shows schematically illustrate an in-ear electrode 76 having a third shape to facilitate positioning of in-ear electrode 76 within ear canal 10 (FIG. 1), according to certain exemplary embodiments.

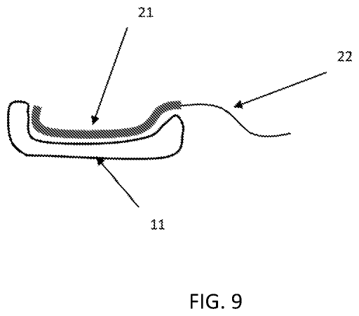

FIG. 9 shows a cross section side view of electrode 21 positioned within the Cavum Conchae 11, according to certain exemplary embodiments. Electrode 21 can include a wire 22 for connecting electrode 21 to a stimulation device or to a second electrode. In some embodiments, electrode 21 can be molded or configured in a concave configuration thereby to facilitating fitting electrode 21 within Cavum Conchae 11. In some embodiments, the geometrical configuration can be non-circular to facilitate fitting the electrode adjacent to the anatomy, and specifically to Cavum Conchae 11.

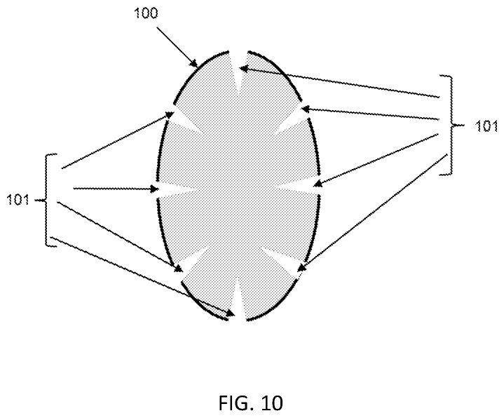

Reference is now made to FIGS. 10A-10B, schematically illustrating an electrode 1M having a plurality of peripheral indentations 110 operative to facilitate positioning of electrode 100 onto a predetermined region of ear 5 (FIG. 1). For example, electrode can include 2, 4, 8, 10 or more peripheral indentations.

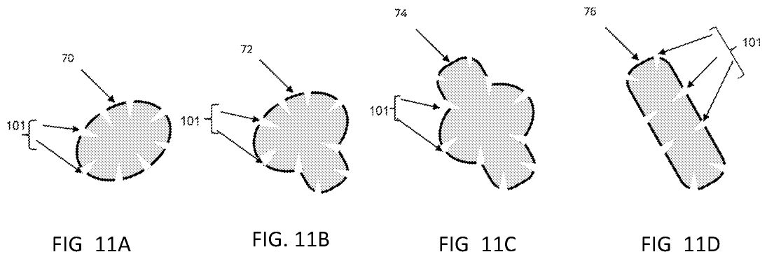

FIGS. 11A-11D schematically illustrate a plurality of in-ear electrode configurations, each in-ear electrode including peripheral indentations to facilitate positioning of the in ear electrode on a concave surface, according to certain exemplary embodiments.

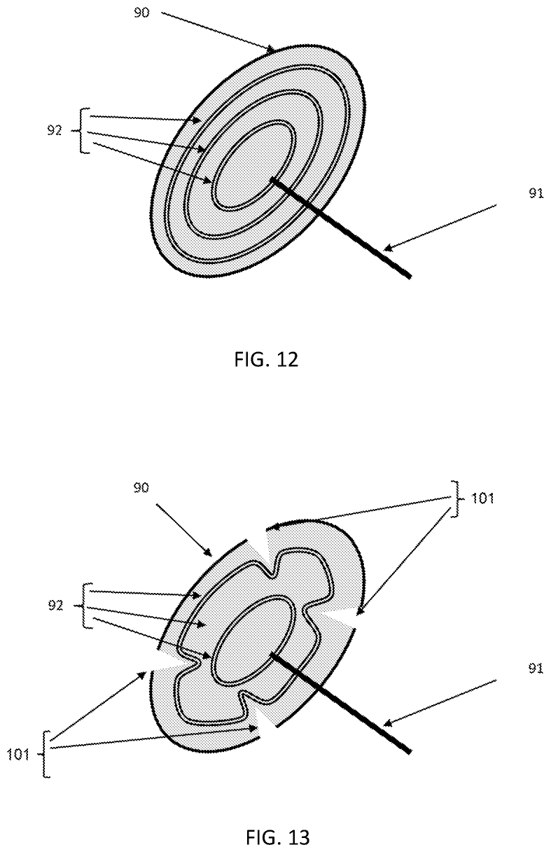

FIG. 12 is a schematic illustration of an in-ear electrode 90 including a uniform current density inner wire net configuration 92, according to certain exemplary embodiment. Uniform current density inner wire net configuration 92 is configured to facilitate distribution of current from electrode 90 to skin in the Concha 11 (FIG. 1A). Uniform current density inner wire net configuration 92 receives an electric current from wire 91.

FIG. 13 is a schematic illustration of in-ear electrode 90 further including peripheral indentations 101, according to certain exemplary embodiments.

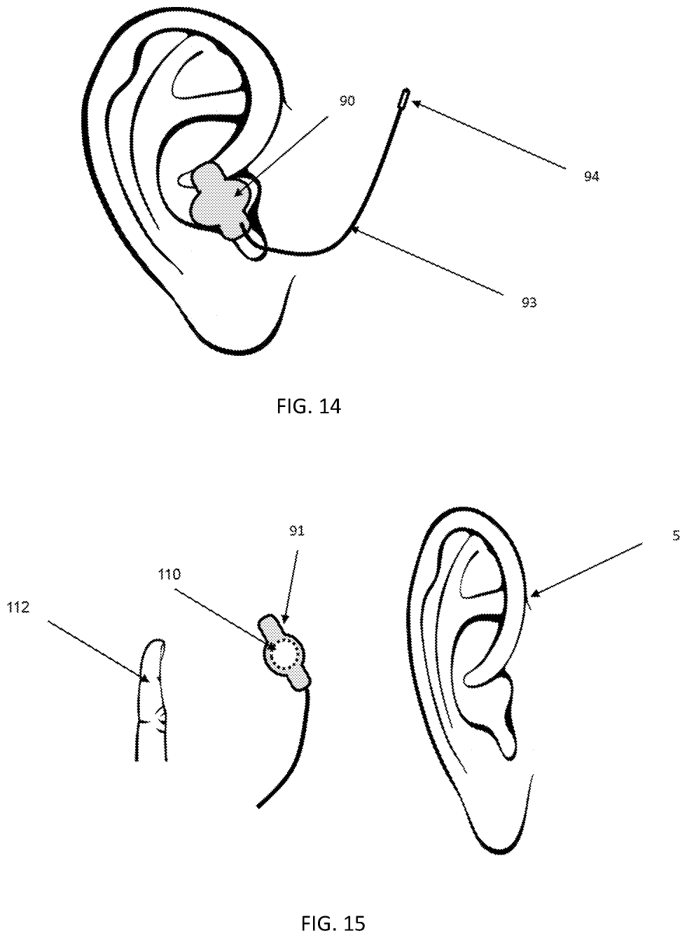

FIG. 14 is a schematic illustration of in-ear electrode 90 including a soft wire 93, which facilitates minimal interference of wire 93 to a subject while the subject is sleeping, according to certain exemplary embodiments. Wire 93 includes a wire connector 94 to facilitate connecting wire 93 and electrode 90 to a power source and a system for operating electrode. In some embodiments, wire 93 is positioned so as to facilitate connection of wire connector 94 to a headband (not shown) of a subject.

FIG. 15 is a schematic illustration of an in-ear electrode 91 further including a finger attachment 110 that facilitates proper positioning of the electrode 91 in the Concha 11 (FIG. 1A), according to certain exemplary embodiments. In some embodiments, finger attachment 110 includes a sticky surface to enable weak adhesion of electrode 91 to a finger 112. The adhesion force of finger attachment 110 is less than an adhesion force of an inner surface area of electrode 91 thereby ensuring that once electrode 91 is connected to a surface of ear 5, finger 112 disconnects from finger attachment 110 without displacement of electrode 91.

In some exemplary embodiments, an electrode 91 is positioned on a surface area of ear 5 after the surface area is cleaned to remove debris, for example with alcohol, after which electrode 91 is positioned on the surface arear. After electrode 91 is positioned on the surface area, for example on Concha 11, electrode 91 is tested to ensure that contact with ear 5 is proper, for example through measuring impedance. In some embodiments, proper positioning is determined when impedance is less than a predetermined value. In some embodiments, electrode 91 can include an adhesive, such as a sticking gel to facilitate connecting electrode 91 to ear 5. Once electrode 91 is properly positioned on ear 5, wire 93 is connected to a stimulator that provides a stimulation current. Stimulator may be connected to the headband on the head of the subject.

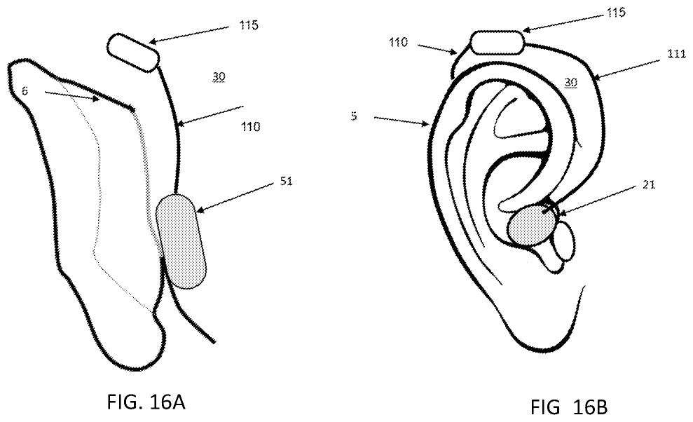

FIGS. 16A-16B are schematic illustrations of an over ear device 115 connected to an in-ear electrode 21 and an out-ear electrode 51, according to certain exemplary embodiments. Over ear device 115 is connected to out-ear electrode 51 and to in-ear electrode with wires 110, 111 respectively. Over ear device 115 can be positioned on a head of user 30.

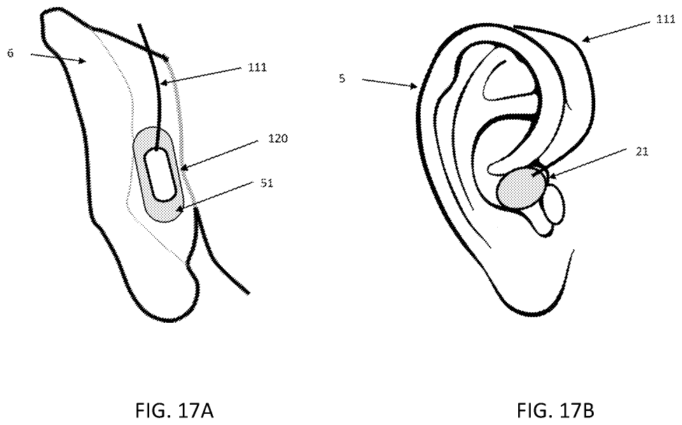

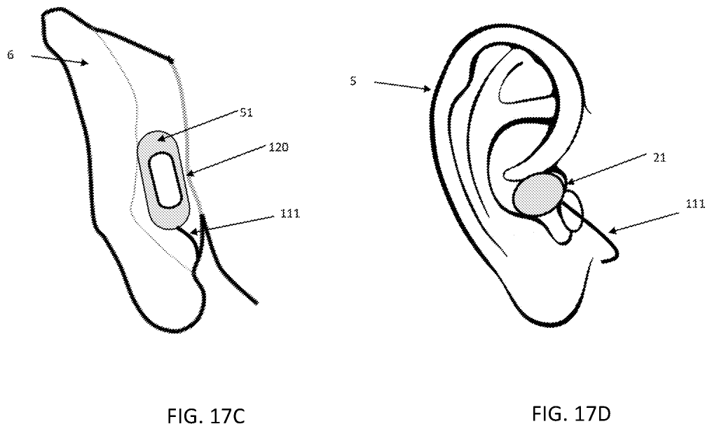

FIGS. 17A-17D are schematic illustrations of a behind the ear device 120, according to certain exemplary embodiments. Behind the ear device 120 is positioned on out-ear electrode 51. In some embodiments, device 120 is connected to in-ear electrode 21 with wire 111 positioned over the ear 5 (FIGS. 17A-1713) and in other embodiments, device 120 is connected to in-ear electrode 21 with wire 111 positioned under the ear 5 (FIGS. 17C-17D).

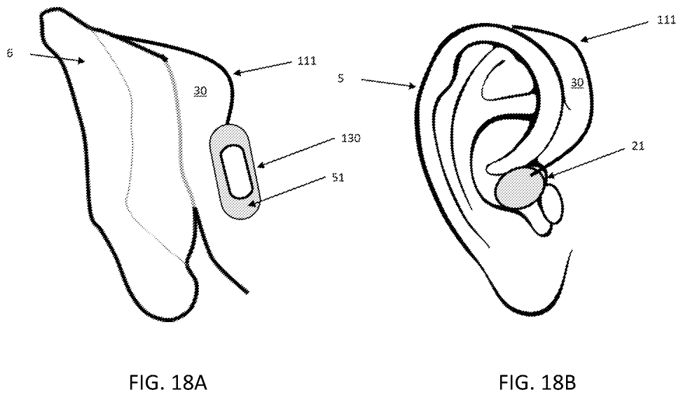

FIGS. 18A-18B are schematic illustrations of a behind the head device 130 connected to out-ear electrode 51 positioned on head 30, according to certain exemplary embodiments.

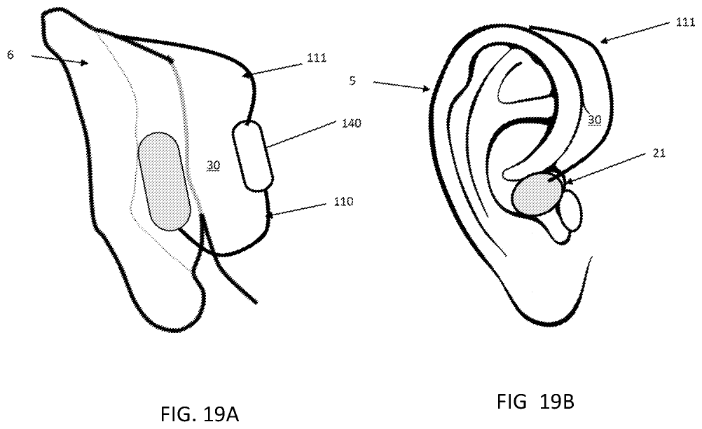

FIGS. 19A-19B are schematic illustrations of a behind the head device 140 connected to in-ear electrode 21 and out-ear electrode 51, according to certain exemplary embodiments. Device 140 is positioned on head 30 adjacent to rear area 6 of ear 5. In some embodiments of this invention, the electrode is constructed from a flexible material, which enables the electrode surface to have maximum skin contact and to achieve the desired nerve stimulation, while reducing potential side effect, such as, stimulation sensation.

In some exemplary cases, the electrode surface that is positioned adjacent to the skin has conductive self-adhesive layer. In specific cases, such layer covers the whole surface of the electrode facing the skin.

In some embodiments, the electrode includes an electrode housing having a how-shaped extension piece designed to be inserted into the auditory canal. In some embodiments, the extension piece includes one electrode contact and the second electrode contact is positioned at the Conchae or other locations at the external ear.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of −10% to +10% of the respective magnitude or value. Unless otherwise specified, the terms ‘about’ and/or ‘close’ with respect to a dimension or extent, such as length, implies within an inclusive range of −10% to +10% of the respective dimension or extent. Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values.

As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded. Terms in the claims that follow should be interpreted, without limiting, as characterized or described in the specification.

1. A device for auricular vagal nerve stimulation in a subject, said device having an electrode configuration comprising:
at least one electrode contact operative to be adhered to a first portion of inner skin surface of an ear Cavum Concha of the subject; and,
at least one return electrode with an opposite polarity operative to be adhered to a second portion of a skin surface of the subject.

2. The electrode configuration according to claim 1, wherein said at least one return electrode comprises a bow shape to facilitate positioning said at least second electrode within the ear canal.

3. The electrode configuration according to claim 1, wherein said at least one return electrode comprises a circular shape to facilitate positioning said at least second electrode within the ear canal.

4. The electrode configuration according to claim 1, wherein said at least one electrode comprises a surface area within a range of 0.5-2 square centimeters.

5. The electrode configuration according to claim 1, wherein said at least one electrode comprises a surface area within a range of 0.1-5 square centimeters.

6. The electrode configuration according to claim 1, wherein said at least one electrode comprises a surface area within a range of 0.2-1 square centimeters.

7. The electrode configuration according to claim 1, wherein said at least one electrode is customizable to fit ear Cavum Concha area.

8. The electrode configuration according to claim 1, wherein said at least first electrode comprises a plurality of peripheral indentations.

9. The electrode configuration according to claim 1, wherein said at least first electrode and said at least second electrode are flexible.

10. The electrode configuration according to claim 1, wherein said at least first electrode is a Transcutaneous electrical nerve stimulation (TENS) electrode.

11. The electrode configuration according to claim 1, wherein said at least second electrode is a Transcutaneous electrical nerve stimulation (TENS) electrode.

12. The electrode configuration according to claim 1, wherein said at least first electrode is operative to provide stimulation while a subject is asleep.

13. The electrode configuration according to claim 1, wherein said second electrode is positioned on the skin surface of rear region of the ear.

14. The electrode configuration according to claim 1, wherein said second electrode is positioned on the skin surface of rear region of a head of the subject.

15. The electrode configuration according to claim 1, wherein said second electrode is positioned on the skin surface at an interface between the ear and a head of the subject.

16. The electrode configuration according to claim 1, wherein said first electrode comprises a surface area greater than 50% of the skin surface of the Cavum Concha.

17. A device for auricular vagal nerve stimulation in a subject, said device having an electrode configuration comprising:
at least one electrode contact operative to be adhered to a first portion of inner skin surface of an ear of the subject; and, at least one return electrode with an opposite polarity operative to be adhered on a skin area of a rear region of the ear.

18. The electrode configuration according to claim 17, wherein said at least one electrode comprises a surface area within a range of 0.1-5 square centimeters.

19. The electrode configuration according to claim 17, wherein said at least one electrode is customizable to fit regions of ear.

20-29. (canceled)

30. A method of positioning an electrode on a skin surface area of an ear, the method comprising:
cleaning a skin surface area;
positioning said electrode on the skin surface area;
measuring an impedance to determine whether said electrode is properly positioned;
connecting said electrode to a stimulator; and, delivering an electrical stimulation to the ear.

31-35. (canceled)
